(19) United States
(12) Patent Application Publication
(10) Pub. No.: US 2010/0081629 A1
(43) Pub. Date: Apr. 1, 2010

(54) LOW MOLECULAR WEIGHT HEPARINS INCLUDING AT LEAST ONE COVALENT BOND WITH BIOTIN OR A BIOTIN DERIVATIVE, METHOD FOR MAKING SAME AND USE THEREOF

(75) Inventors: Christian Viskov, Ris Orangis (FR); Philippe Hubert, Maisons-Alfort (FR); Pierre Mourier, Charenton LePont (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/539,224

(22) Filed: Aug. 11, 2009

Related U.S. Application Data
(63) Continuation of application No. PCT/FR2008/000173, filed on Feb. 12, 2008.

(30) Foreign Application Priority Data
Feb. 14, 2007 (FR) 0701055

(51) Int. Cl. A61K 31/727; C07H 1/00

(52) U.S. Cl. 514/56; 536/123.1

(57) ABSTRACT

The invention relates to biotinylated low molecular weight heparins comprising constituent polysaccharides having at their reducing ends at least one covalent bond with biotin or a biotin derivative, and also to the process for preparing them, to pharmaceutical compositions containing them and to their therapeutic use.

### FIELD OF THE INVENTION

The present invention relates to low molecular weight heparins, more generally heparinoid-based polysaccharide mixtures, which contain at least one covalent bond with biotin or a biotin derivative, and also to the process for preparing them, to pharmaceutical compositions containing them and to their therapeutic use.

### BACKGROUND OF THE INVENTION

Heparin is a mixture of sulfated mucopolysaccharides of animal origin, with a molecular weight in the region of 15 000 daltons (Da), used especially for its anticoagulant and antithrombotic properties. However, heparin has drawbacks that limit its conditions of use. In particular, its high anticoagulant activity (especially its high anti-factor IIa activity) may cause hemorrhaging (Seminars in Thrombosis and Hemostasis, vol. 5, sup. 3, 1999).

Low molecular weight heparins between, for example, 3000 and 7000 Da and more particularly between 3500 and 5500 daltons, obtained especially by basic depolymerization of heparin esters and currently marketed, such as enoxaparin, also have high anti-factor IIa activity.

Heparin derivatives are known for these undesirable hemorrhagic side effects. However, in the field of treating thrombosis with the above products, the aim is to reestablish or maintain blood fluidity while at the same time avoiding the induction of a hemorrhage. In point of fact, it is well known that, for any accidental reason, a hemorrhage may be triggered in a patient under treatment. There may also be a need to perform a surgical operation on a patient under antithrombotic treatment. Furthermore, in the course of certain surgical operations, anticoagulants may be used at high dose so as to prevent coagulation of the blood, and it would be useful to be able to neutralize them at the end of the operation. There is thus a need for neutralizable antithrombotic agents to stop the anticoagulant activity at any moment.

Neutralizable antithrombotic agents, such as biotinylated synthetic polysaccharides, have been described in patent applications WO 02/24754 and WO 06/030 104. Their synthesis, especially comprising the grafting of biotin or of the biotin derivative performed on protected equivalents of the polysaccharides mentioned above rather than on these polysaccharides themselves, is not applicable to the compounds of the present invention. The reason for this is that it is desired to perform the biotinylation on finished products, which are mixtures of heparin-based polysaccharides and are thus heterogeneous products, on which the grafting of biotin as described in the abovementioned patent applications would not make it possible to induce a sufficient regioselectivity of the grafting position and would not allow biotinylation of all the functionalizable polysaccharide chains of low molecular weight heparins.

The team of Osmond et al. describes, in Analytical Biochemistry, 31 (2002) 199-207, several techniques for biotinylating a porcine heparin, one of them being described as involving a coupling of biotin at the reducing end of a heparin via a reductive amination followed by coupling with biotin. However, the operating conditions described in the said document do not allow biotinylated heparins to be obtained fully and reproducibly: they do not take into account the structural diversity of heparins and the real structure of the polysaccharide chains as present in the commercially available heparins. The latter heparins comprise a large proportion of polysaccharide chains that contain at their reducing end a degraded glycoserine, which is not functionalizable with biotin according to the protocol described by Osmond et al. Thus, the operating conditions described in the said publication for the biotinylation of porcine heparin do not allow biotinylated heparins to be obtained fully and reproducibly with expected characteristics, such as a degree of biotinylation sufficient to allow efficient neutralization.

The team of Tseng et al. describes, in Biomaterials, 27 (2006), 2627-2636, a technique for immobilizing heparin on films by interaction with avidin, following functionalization of the heparin with biotin. The biotinylation of heparin is performed via oxidation with iodine, followed by the formation of a lactone, and then coupling with a biotin 2-(4-aminophenyl)ethylamine derivative. The operating conditions presented by Tseng et al. do not, however, assume the total and reproducible production of heparins biotinylated at the reducing end: specifically, there is nothing to indicate that the oxidation step may be selective on the reducing end, or that the biological activity of heparin is conserved after such a treatment.

The Applicant thus set itself the aim of providing novel low molecular weight heparins that can be neutralized with avidin or streptavidin and that have biological properties, especially anti-factor Xa and anti-factor IIa activities, comparable to the starting low molecular weight heparins.

### SUMMARY OF THE INVENTION

The present invention relates to novel modified low molecular weight heparins, referred to hereinbelow as “biotinylated low molecular weight heparins”, characterized in that they have an average molecular weight of between 3000 and 7000 Da and in that their constituent polysaccharides are covalently bonded to biotin or a biotin derivative at their reducing end.

Surprisingly, the introduction of biotin or of a biotin derivative at the reducing end of the polysaccharide chains does not modify the pharmacological activity of the low molecular weight heparins. Specifically, the novel biotinylated low molecular weight heparins that are the subject of the invention have antithrombotic activities comparable to native low molecular weight heparins, i.e. heparins before biotinylation.

They have a considerable advantage over native low molecular weight heparins: they may be rapidly neutralized with a specific antidote, in the case of emergency. This specific antidote is avidin, in tetrameric or monomeric form, or streptavidin, with respective masses equal to about 66 000, 16 400 and 60 000 Da (The Merck Index, Twelfth edition, 1996, M.N. 920, pages 151-152, Revue Pierce Avidin-Biotin Handbook).

They also have the advantage of being useful in therapeutic indications for which the doses used are higher, while at the same time reducing the risk of hemorrhage; they may thus be useful in the arterial therapeutic field.

### DESCRIPTION OF THE DRAWINGS

FIG. 1 is a graphical representation which illustrates the reaction monitoring by HPLC SAX of the conversion of enoxaparin according to Example 1.

FIGS. 2, 3 and 4 are graphical representations which illustrate the analyses by HPLC SAX of the biotinylated and non-biotinylated fractions obtained after passing the products obtained according to Examples 1, 2 and 3, respectively, through a supported avidin monomer column.

### DETAILED DESCRIPTION OF THE INVENTION

In the context of the present invention, the term “low molecular weight heparins” means mixtures of sulfated polysaccharides that have the general structure of the constituent polysaccharides of heparin, which have an average molecular weight of from 3000 to 7000 Da and which are obtained by depolymerization of heparin. In the text hereinbelow, the term “low molecular weight heparins” or “native low molecular weight heparins” denotes polysaccharide mixtures before biotinylation, in contrast with the term “biotinylated low molecular weight heparins”, which denotes the compounds according to the invention, comprising a covalent bond to biotin or a derivative thereof.

The term “reducing end” means the end of the polysaccharide chain in which the terminal glucosamine or mannosamine (mannosamine resulting from an epimerization in basic medium of glucosamine) has a cyclic hemiacetal function, corresponding to formula (II) below:

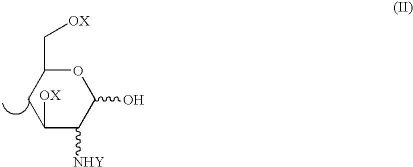

in which
X represents H or SO₃Na,
Y represents COCH₃ or SO₃Na, and
the wavy line denotes a bond located either below or above the plane of the pyranose ring to which it is attached (below: glucosamine, above: mannosamine).

Among the low molecular weight heparins that may be used in the present invention, some of them may be such that at least 75% of their polysaccharide chains comprise at their reducing end a glucosamine in hemiacetal form; these are the functionalizable polysaccharides of the mixture. Certain polysaccharide chains present in the mixture may be in 1,6-anhydro form, to a content of less than or equal to 25%; such polysaccharides are not functionalizable with biotin or the biotin derivative.

The term “constituent functionalizable polysaccharides” of the mixture means the polysaccharides comprising at their reducing end a glucosamine in hemiacetal form as defined in formula (II).

The term “constituent polysaccharides of heparin” means polysaccharides characterized by the repetition of a disaccharide unit containing a uronic acid residue (D-glucuronic acid or L-iduronic acid) and a D-glucosamine residue, which may be N-sulfated or N-acetylated. The disaccharide unit may also be O-sulfated in positions C6 and/or C3 of D-glucosamine and in position C2 of uronic acid (Heparin-binding proteins, H. Edward Conrad, 1998, p. 1).

The biotinylated low molecular weight heparins according to the invention are advantageously characterized in that their constituent polysaccharides correspond to the general formula (I):

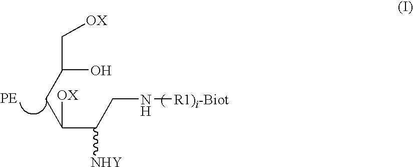

in which:
i is equal to 0 or 1,
R1 represents a sequence of formula (a) or (b):

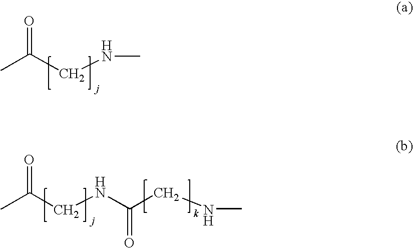

in which j and k, which may be identical or different, are integers that may take any value from 1 to 10,
Biot represents a biotin group or biotin derivative,
PE represents a polysaccharide chain having the general structure of the constituent polysaccharides of heparin,
X represents H or SO₃Na,
Y represents SO₃Na or COCH₃,
the wavy line denotes a bond located either below or above the plane of the pyranose ring to which it is attached,
   and also the pharmaceutically acceptable salts thereof.

The biotin (Biot) group mentioned above is a radical derived from hexahydro-2-oxo-1H-thieno[3,4-d]imidazole-4-pentanoic acid. Advantageously, the Biot group in the general formula (I) according to the invention corresponds to formula (c):

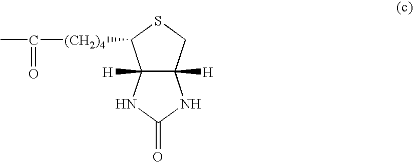

The biotin derivatives are commercially available (“Pierce” Biotin-avidin products catalogue, 2005, pp. 7-11) or may be prepared using standard methods known to those skilled in the art. Mention may be made especially of the biotin derivatives mentioned in patent application WO 02/24754.

In the biotinylated low molecular weight heparins according to the invention, the index i may be equal to 0, in which case the bond with biotin or the biotin derivative is made directly on the amine function borne by the saccharide unit on the reducing end of the polysaccharide chains.

Alternatively, i may be equal to 1 and the bond with the biotin group or biotin derivative may consist, for example, of a sequence of formula (a) above in which j is equal to 5, or of a sequence of formula (b) above in which j and k are identical and are equal to 5. Thus, in formula (I) above, R1 may represent, for example, a sequence of formula —CO—(CH₂)₅—NH or —CO—(CH₂)₅—NH—CO—(CH₂)₅—NH—.

The biotinylated low molecular weight heparins according to the present invention are such that at least 60%, advantageously at least 80% and even more advantageously at least 90% of their constituent polysaccharides have at their reducing end a covalent bond to biotin or a biotin derivative.

The low molecular weight heparins used in the present invention may be chosen, for example, from enoxaparin, ardeparin, bemiparin, parnaparin and tinzaparin.

As defined in U.S. Pat. No. 5,389,618 and U.S. RE38,743, the low molecular weight heparins used in the present invention may especially be such that:
from 9% to 20% of their constituent polysaccharides have an average molecular weight of less than 2000 Da,
from 5% to 20% of their constituent polysaccharides have an average molecular weight of greater than 8000 Da,
from 60% to 86% of their constituent polysaccharides have an average molecular weight of between 2000 and 8000 Da,
the ratio between the mass-average molecular mass and the number-average molecular mass is between 1.3 and 1.6, and
the said low molecular weight heparins have better bioavailability and antithrombotic activity than that of heparin and have an average molecular weight of approximately between 3500 and 5500 Da.

The invention covers biotinylated low molecular weight heparins in the form of any of their pharmaceutically acceptable salts.

A subject of the present invention is also a process for preparing the biotinylated low molecular weight heparins mentioned above, characterized in that:

a) a reductive amination is performed, in the presence of an amine salt and a reducing agent and at a temperature of between 20 and 80° C., on a low molecular weight heparin as defined above,
b) an acylation is then performed with an activated group —(R1)ᵢ-Biot, in which R1, i and Biot are as defined in relation with formula (I) above, in the presence of a base in aqueous medium or in organic medium.

The steps of the above preparation process may be controlled by analytical HPLC monitoring, especially of SAX type, using, for example, the method described in patent application WO 2004/027087, or optionally via LC-MS, using, for example, the method described by Robert J. Linhardt in J. Biol. Chem., 2004, 279 (4), p. 2608-2615.

The biotinylated low molecular weight heparins may also be analyzed and characterized by affinity chromatography on supported monomeric avidin, sold by the company Pierce, according to the analytical conditions described by the supplier.

It is especially confirmed, after the reductive amination step a), that at least 90% of the constituent polysaccharides of the said low molecular weight heparins bear at their reducing end an —NH₂ function (amino-reduced polysaccharides).

It is especially confirmed, after the acylation step b), that at least 90% of the said amino-reduced polysaccharides are biotinylated.

The overall yield of the process for preparing the biotinylated low molecular weight heparins according to the invention is thus at least 80% and advantageously at least 90%.

The process for preparing the compounds according to the invention uses as starting low molecular weight heparins (“native” low molecular weight heparins) low molecular weight heparins prepared as reported previously in the literature. Reference will be made especially to U.S. Pat. RE38,743 for enoxaparin, U.S. Pat. No. 4,757,057 for ardeparin, EP 0 293 539 for bemiparin, U.S. Pat. No. 4,791,195 for parnaparin and U.S. Pat. No. 5,106,734 for tinzaparin.

In the reductive amination step a) of the above preparation process, the amine salt may be a quaternary amine salt; it is advantageously an ammonium halide salt corresponding to the formula NH₄Z, in which Z represents a halogen atom, such as a chlorine, fluorine, bromine or iodine atom.

In the reductive amination step a) of the above preparation process, the reducing agent may be a borohydride salt, for example a cyanoborohydride salt.

In the reductive amination step a) of the above preparation process, the temperature is advantageously between 50 and 80° C.

In the acylation step b) of the above preparation process, the base may be a carbonate or hydrogen carbonate salt, especially in sodium or potassium salt form, or alternatively any water-soluble or organo-soluble organic base known to those skilled in the art.

In the acylation step b) of the above preparation process, the term “organic medium” means, for example, dichloromethane or dimethylformamide.

The process for preparing the biotinylated low molecular weight heparins according to the invention advantageously comprises the following steps:

a) a reductive amination is performed on a low molecular weight heparin in the presence of an ammonium halide salt and a borohydride salt, at a temperature of between 50 and 80° C.,
b) an acylation is then performed with a group —(R1)ᵢ-Biot as defined above in activated ester form, in the presence of a base in aqueous medium.

The biotinylated derivatives —(R1)ᵢ-Biot as defined above may be used in the acylation reaction directly in the form of activated esters, preformed or generated in situ using standard coupling conditions known to those skilled in the art. Activated esters in the form of N-hydroxysuccinimide derivatives or of 3-sulfo-N-hydroxy-succinimide derivatives may especially be used.

The preparation process according to the invention is illustrated in Scheme 1.

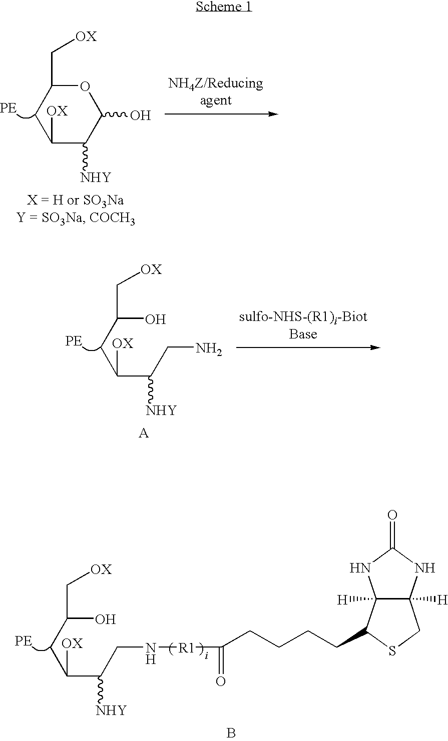

According to Scheme 1, the low molecular weight heparin is subjected to a reductive amination to produce derivative A, containing a free amine function at the reducing end, in the presence of an amine salt and a reducing agent such as a borohydride salt.

This derivative may then be acylated to provide the biotinylated derivative B, via reaction with an activated biotin derivative —(R1)ᵢ-Biot, as defined above, in the presence of a base. This reaction may be performed, for example, with the sodium salt of the ester 3-sulfosuccinimidyl 6-biotinamidohexanoyl hexanoate when R1 represents the sequence —CO—(CH₂)₅—NH—CO—(CH₂)₅—NH—, or with the sodium salt of the ester 3-sulfosuccinimidyl 6-biotinamido hexanoate when R1 represents the sequence —CO—(CH₂)₅—NH—, or alternatively with the sodium salt of the biotinoyl-3-sulfosuccinimidyl ester when R1 is not present (i=0).

In Scheme 1, it is understood that the derivatives A and B are a theoretical representation, since it is a matter in reality, as low molecular weight heparin derivatives of mixtures of polysaccharide chains.

In the text hereinbelow, examples of synthesis of the biotinylated low molecular weight heparins according to the invention and of various intermediates that are useful for obtaining them are detailed by way of illustration.

The following abbreviations are used:

HPLC: high-performance liquid chromatography;
SAX: strong anion exchange chromatography;
LC-MS: liquid chromatography-mass spectroscopy;
qs: quantity sufficient;
LC: long chain, corresponding to the 6-aminohexanoyl sequence;
LC-LC: represents two LC sequences and corresponds to the amido-hexanoyl-6-aminohexanoyl sequence;
sulfo-NHS: sodium salt of the 3-sulfosuccinimidyl ester;
Heparinase 1: heparin lyase I enzyme (EC 4.2.2.7) from Flavobacterium heparinum

### Example 1

### NH LC Biotinoyl Enoxaparin

Enoxaparin is a low molecular weight heparin obtained according to the process described in U.S. Pat. RE38,743. It is converted into the biotinylated derivative according to the reaction sequence in Scheme 2: enoxaparin is converted via a reductive amination reaction into compound 1 having an amino function at its reducing end, and this derivative is then converted into the biotinylated compound 2 via reaction with 3-sulfosuccinimidyl 6-biotinamido hexanoate, sodium salt.

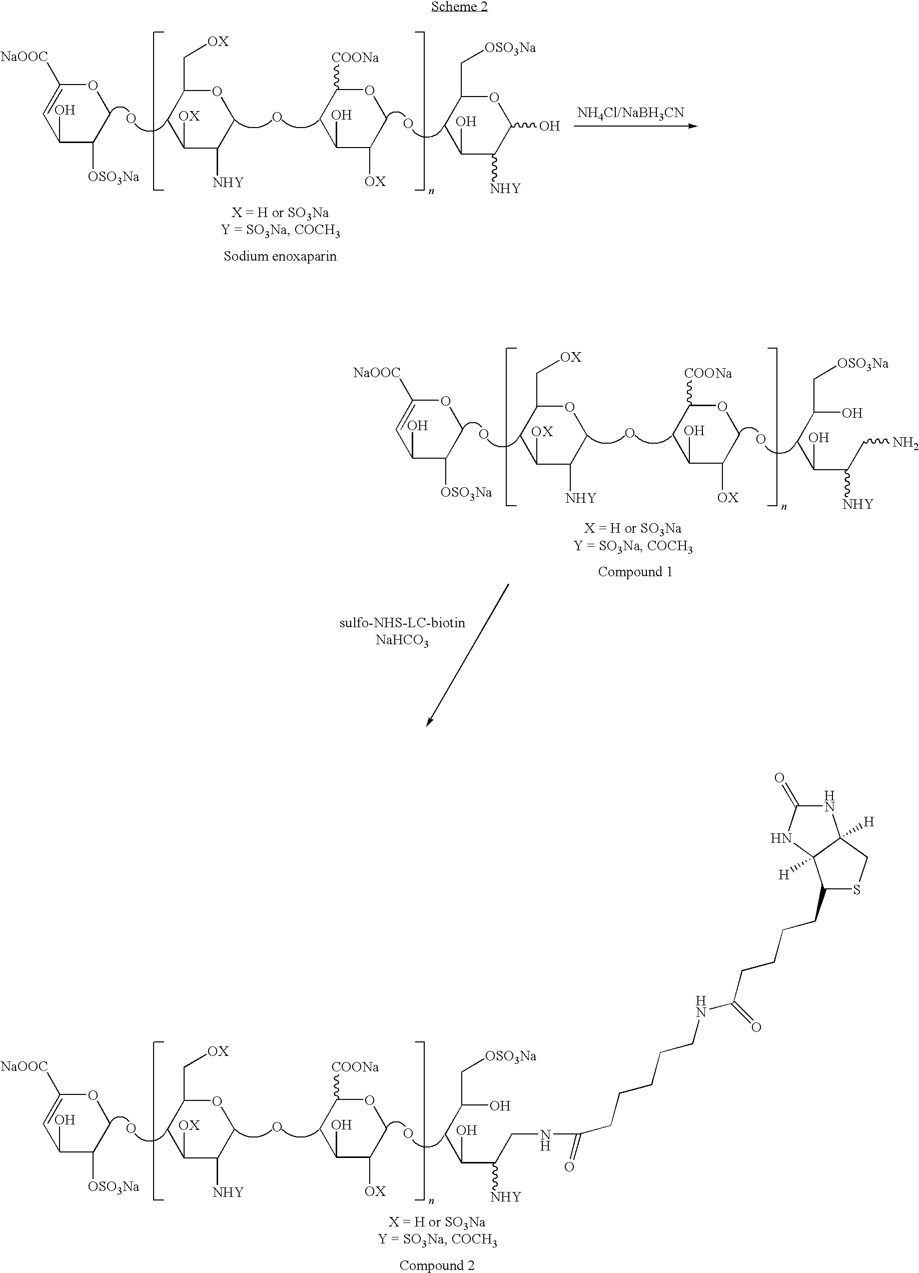

### 1.1: 1-Amino Enoxaparin

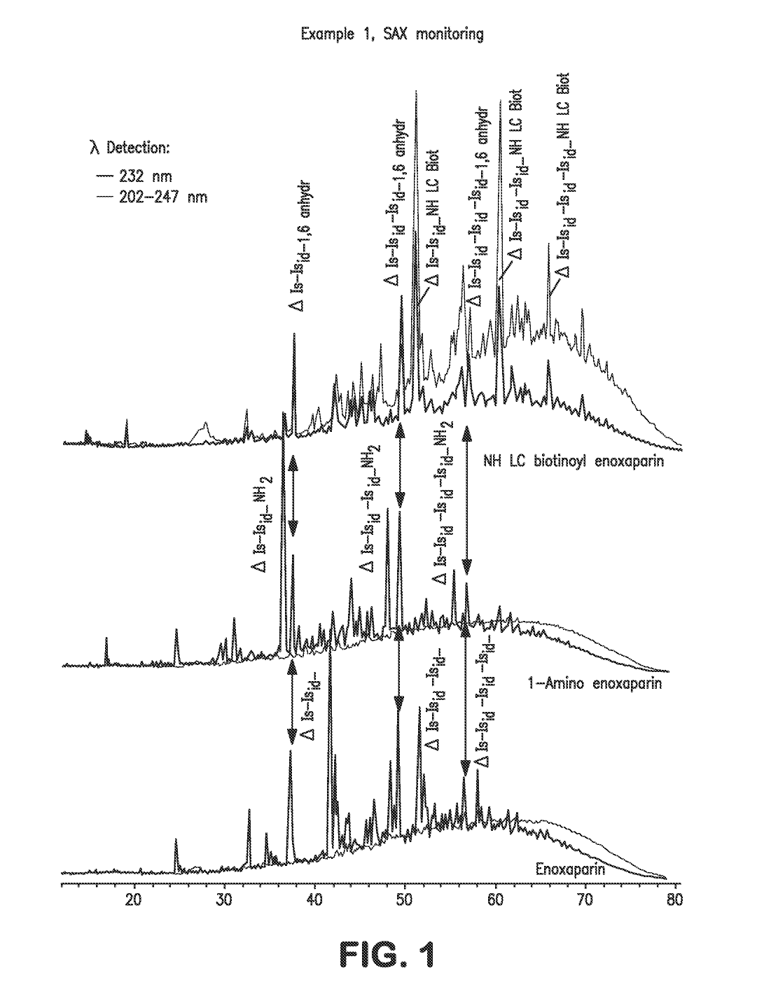

1 g of enoxaparin is dissolved in 40 ml of aqueous 5 M ammonium chloride solution. 1 g of sodium cyanoborohydride is added to the solution obtained. The mixture is maintained at 60° C. for 24 hours. The solution is cooled to a temperature in the region of 20° C. and diluted with water (qs 100 ml). The filtrate obtained is desalified on a column of Sephadex G10 and then freeze-dried. 824 mg of a white lyophilizate are obtained. The observed yield is 82%. The product is controlled by HPLC SAX (see FIG. 1) and used without further purification in the biotinylation step.

### 1.2: NH LC Biotinoyl Enoxaparin

200 mg of 1-amino enoxaparin are dissolved in 5 ml of 0.5 M sodium hydrogen carbonate solution at a temperature in the region of 20° C. 136 mg of sulfo-NHS-LC-biotin are added to the solution obtained. The solution is stirred at a temperature in the region of 20° C. for 1 hour. The suspension obtained is diluted with 10 ml of 0.5 M sodium hydrogen carbonate solution. 136 mg of sulfo-NHS-LC-biotin are added and the mixture obtained is stirred for 18 hours. A further 136 mg of sulfo-NHS-LC-biotin are added and the reaction mixture is stirred for 1 hour. A further 70 mg of sulfo-NHS-LC-biotin are added and the reaction mixture is stirred for 3 hours. The reaction medium obtained is diluted with water (qs 200 ml), filtered on a 0.45 μm membrane and then desalified on a column of Sephadex G10. The fraction obtained is injected onto a Q-Sepharose column. The product is eluted with water and then with a gradient of sodium perchlorate. The collected fraction is desalified on a column of Sephadex G10. The product obtained is again purified by passing it through a column of Q-Sepharose and desalifying on Sephadex G10. The final fraction collected is freeze-dried. 190 mg of a white lyophilizate are obtained. The observed yield is 87%.

¹H NMR spectrum of the mixture of oligosaccharides in D₂O (25° C., δ in ppm): between 1.3 and 1.8 (12H, m), 2.05 (OH₃CO, s), 2.25 (2CH₂CO biotin, m), 2.80 (1H, d, 12 Hz), 3.03 (1H, dd, 12 and 5 Hz), between 3.15 and 5.65 (polysaccharide protons), 5.99 (1H, d, 4 Hz).

The product obtained is controlled by HPLC SAX: FIG. 1 (drawing 1/4) illustrates the reaction monitoring by HPLC SAX of the conversion of enoxaparin via a reductive amination reaction to derivative 1 having an amino function on its reducing end (cf. Scheme 2). This derivative is then converted into the biotinylated derivative 2 via reaction with 3-sulfosuccinimidyl 6-biotinamido hexanoate, sodium salt. The analytical method used is described in patent application WO 2004/027087. FIG. 1 shows that the species containing a functionalizable glucosamine are converted into derivatives containing an amino function on their reducing end with a degree of conversion of greater than 90% to give 1-amino enoxaparin. FIG. 1 also shows that the species containing an amino function on their reducing end are converted into the biotinylated derivative via reaction with 3-sulfosuccinimidyl 6-biotinamido hexanoate, sodium salt, with a degree of conversion of greater than 90% to give NH LC biotinoyl enoxaparin.

By way of example, FIG. 1 indicates the peaks corresponding to the main compounds present in the oligosaccharide mixtures obtained according to Example 1, the structure of which is represented below (the nomenclature used corresponds to that of patent application WO 2004/027087).

LC-MS analysis allows confirmation of the structure of these compounds via the mass spectra corresponding to the products in acid form: ΔIsIs_{Id} m/z=1154; ΔIsIs_{Id}Is_{Id} m/z=1731; ΔIsIs_{Id}Is_{Id}Is_{Id} m/z=2308; ΔIsIs_{Id1,6-anhydro} m/z=1056; ΔIsIs_{Id}Is_{Id1,6-anhydro} m/z=1633; ΔIsIs_{Id}Is_{Id}Is_{Id1,6-anhydro} m/z=2210; ΔIsIs_{Id} NH₂ m/z=1155; ΔIsIs_{Id}Is_{Id} NH₂ m/z=1732; ΔIsIs_{Id}Is_{Id}Is_{Id} NH₂ m/z=2309; ΔIsIs_{Id} NH LC Biot m/z=1494; ΔIsIs_{Id}Is_{Id} NH LC Biot m/z=2071; ΔIsIs_{Id}Is_{Id}Is_{Id} NH LC Biot m/z=2648.

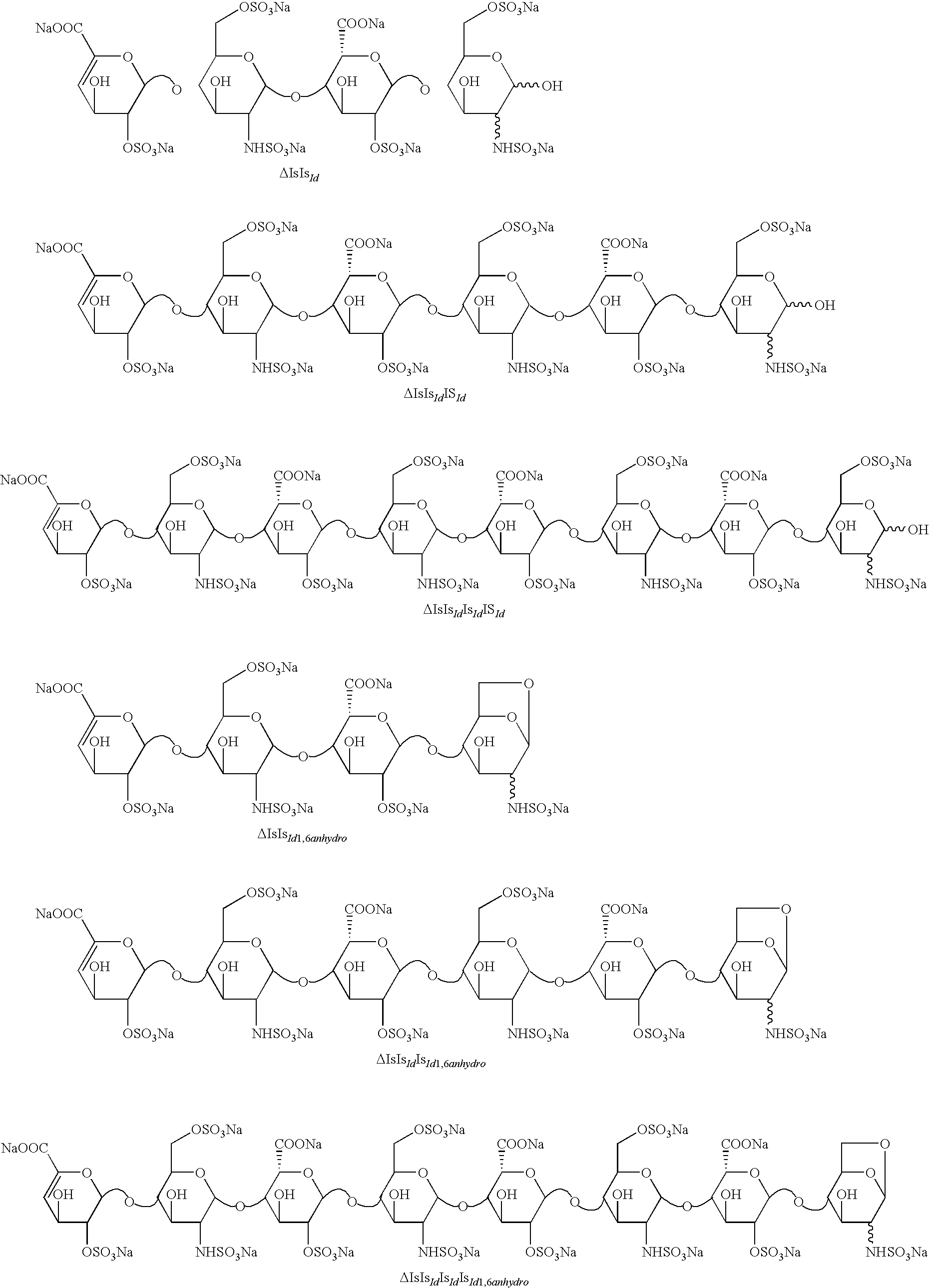

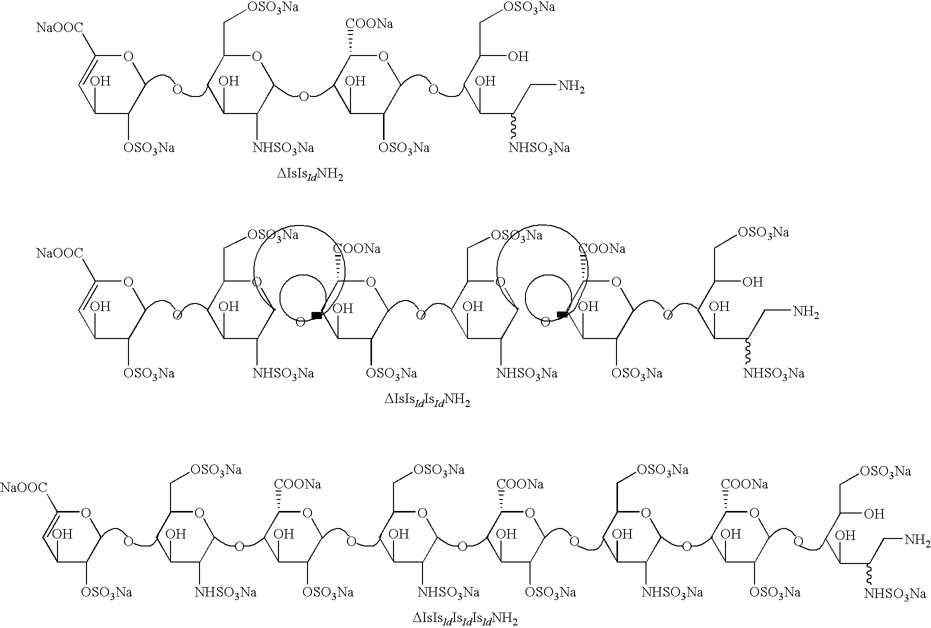

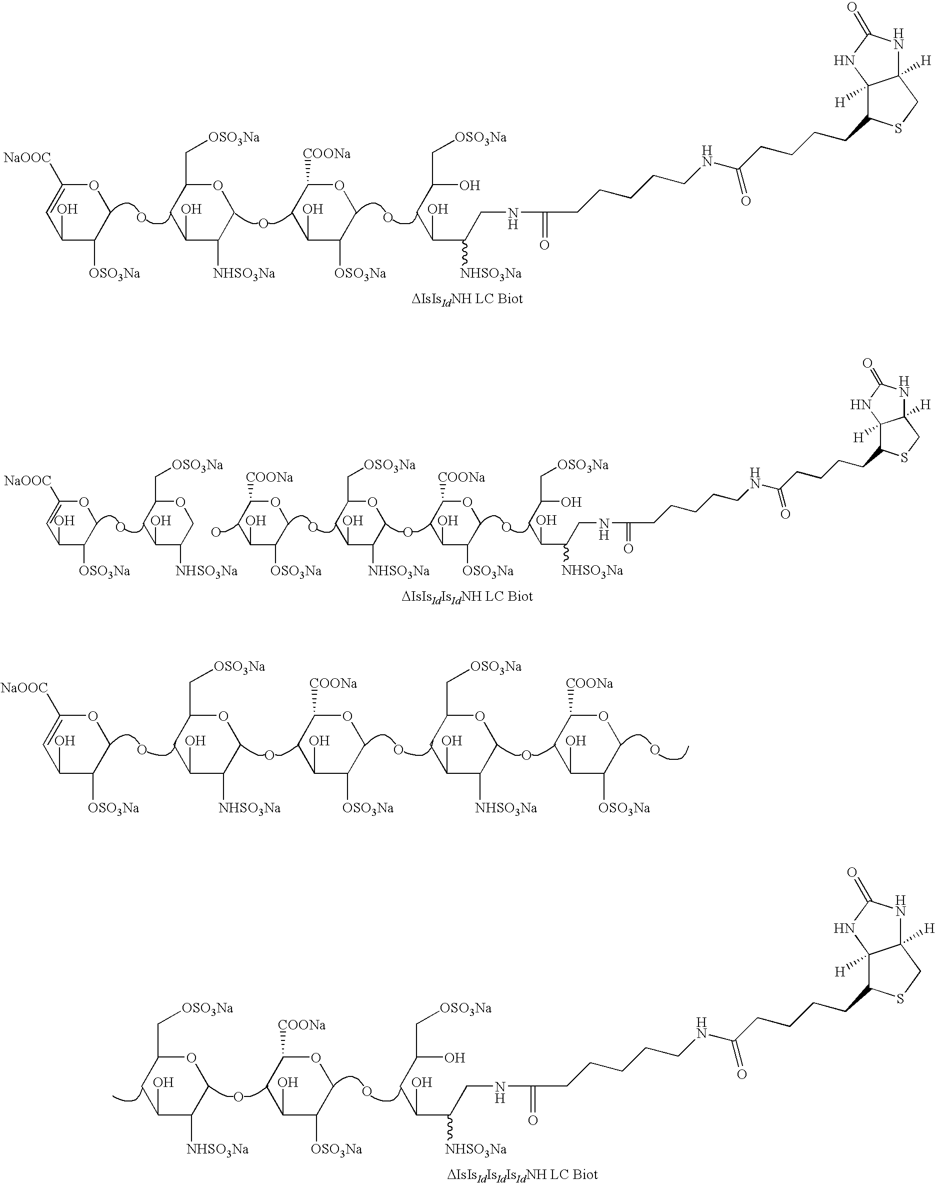

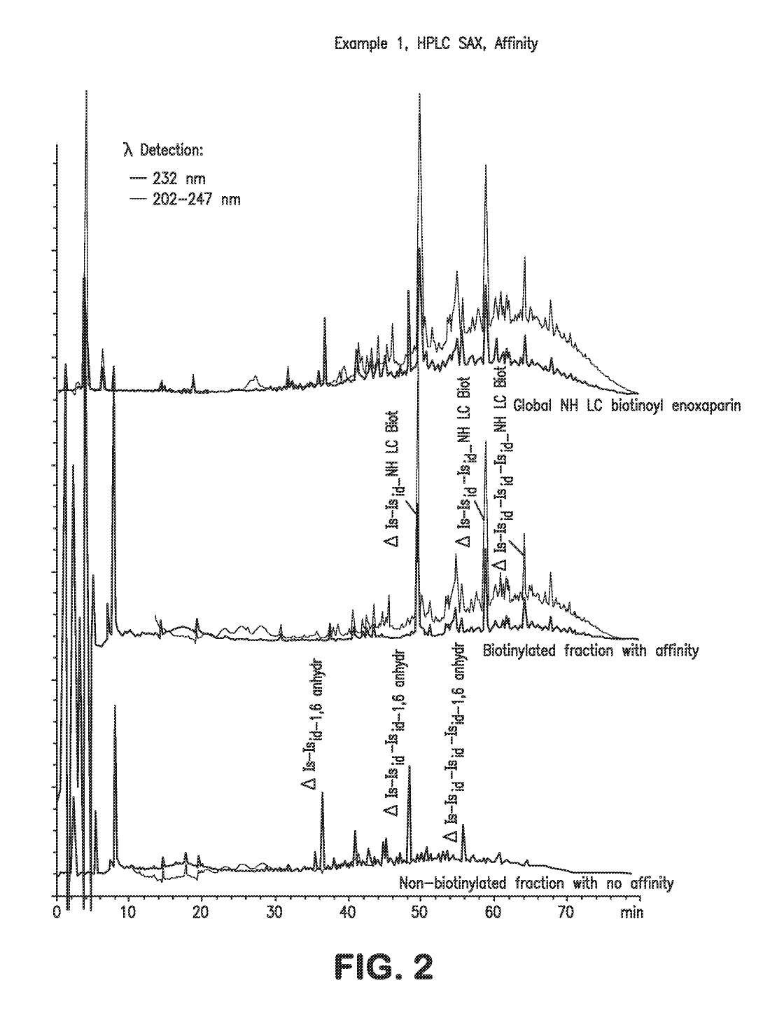

Moreover, the product obtained according to Example 1 may be injected onto a supported avidin monomer column. Elution is performed according to the conditions described by the supplier Pierce. The biotinylated fractions (with affinity for avidin) and the non-biotinylated fractions (with no affinity for avidin) thus obtained are then injected onto HPLC SAX (see FIG. 2, drawing 2/4): FIG. 2 illustrates the HPLC SAX analysis of the biotinylated and non-biotinylated fractions obtained after passage through the supported avidin monomer column. FIG. 2 shows that the species containing a functionalizable glucosamine have been converted into corresponding biotinylated species with a degree of conversion of greater than 90%. The fraction with no affinity is constituted mainly of 1,6-anhydro derivatives, which, by their nature, cannot be converted into biotinylated derivatives. The structures of some of the main peaks are given by way of example to characterize the product obtained (cf. structures illustrated above).

### Example 2

### NH Biotinoyl Enoxaparin

Enoxaparin, a low molecular weight heparin obtained according to the process described in U.S. Pat. RE38,743, is converted into the biotinylated derivative according to the reaction sequence described in Scheme 3: enoxaparin is converted via a reductive amination reaction into compound 1 containing an amino function at its reducing end, and this derivative is then converted into the biotinylated compound 3 via reaction with the biotinoyl-3-sulfosuccinimidyl ester, sodium salt.

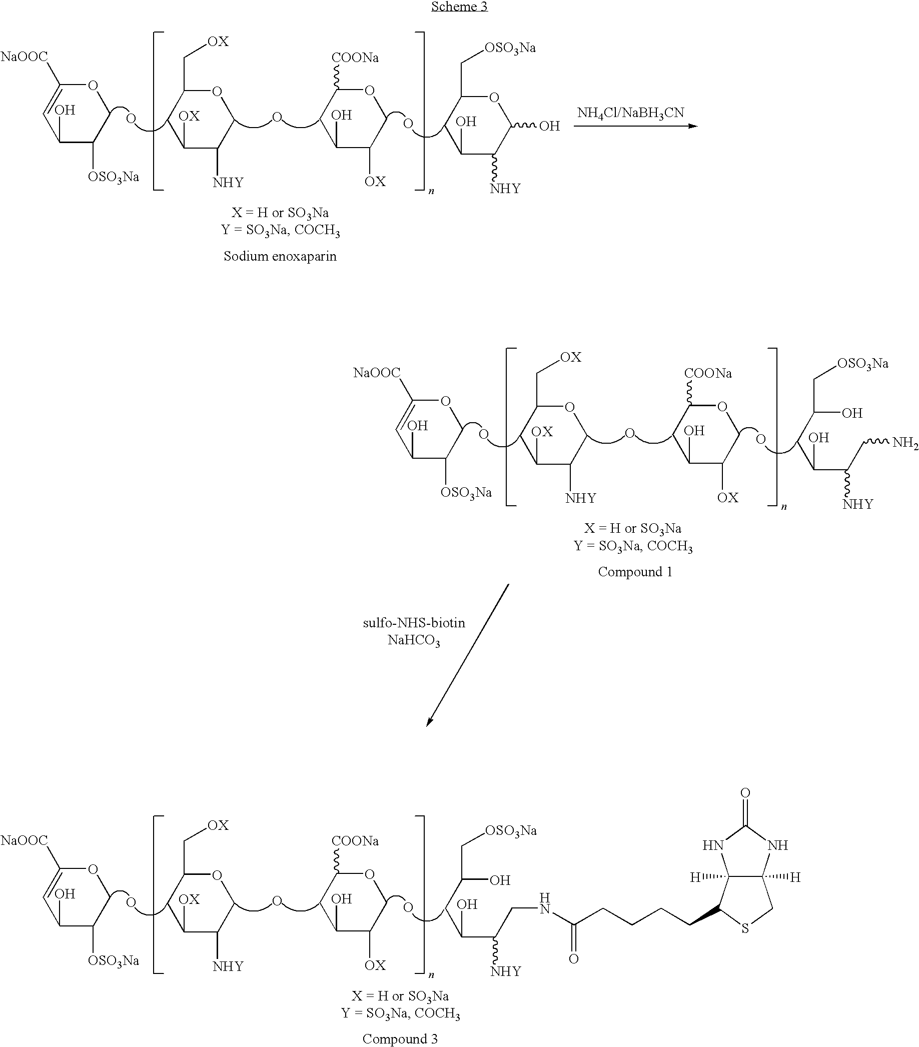

200 mg of 1-amino enoxaparin are dissolved in 5 ml of 0.5 M sodium hydrogen carbonate solution at a temperature in the region of 20° C. 107 mg of sulfo-NHS-biotin are added to the solution obtained. The solution is stirred at a temperature in the region of 20° C. for 1 hour 30 minutes. The suspension obtained is diluted with 10 ml of 0.5 M sodium hydrogen carbonate solution. 107 mg of sulfo-NHS-biotin are added and the mixture obtained is stirred for 3 hours. The reaction medium obtained is diluted with water (qs 150 ml), filtered through a 0.45 μm membrane and then injected onto a column of Q-Sepharose. The product is eluted with water and then with a gradient of sodium perchlorate. The collected fraction is desalified on a column of Sephadex G10. The collected fraction is freeze-dried. 190 mg of a white lyophilizate are obtained. The observed yield is about 90%.

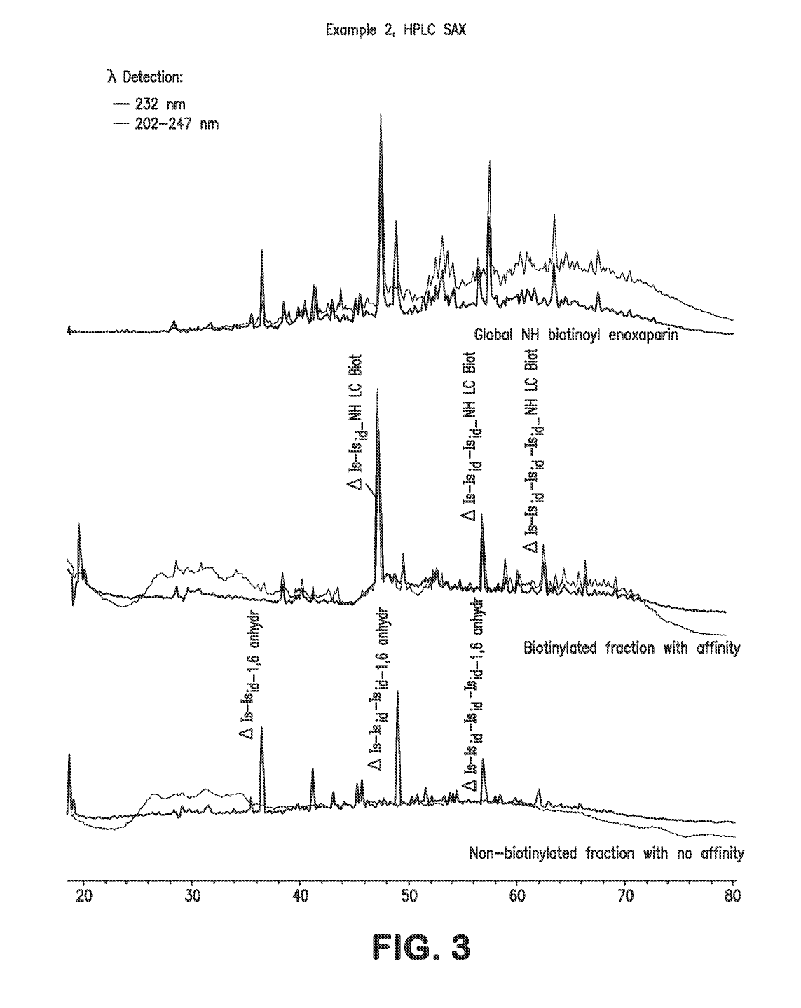

The product obtained is controlled by HPLC SAX (see FIG. 3, drawing 3/4, “Global” graph) and it is confirmed that the species containing an amino function on their reducing end are converted into the biotinylated derivative via reaction with the biotinoyl-3-sulfosuccinimidyl ester, sodium salt, with a degree of conversion of greater than 90%.

¹H NMR spectrum of the mixture of oligosaccharides in D₂O (25° C., δ in ppm): between 1.4 and 1.8 (6H, m), 2.05 (CH₃CO, s), 2.3 (CH₂CO biotin, m), 2.80 (1H, dd, 12 and 7 Hz), 3.03 (1H, m), between 3.20 and 5.65 (polysaccharide protons), 5.98 (1H, d, 4 Hz).

The product obtained according to Example 2 is injected onto a supported avidin monomer column. Elution is performed according to the conditions described by the supplier Pierce. The biotinylated fractions (with affinity for avidin) and non-biotinylated fractions (with no affinity for avidin) obtained are then injected onto HPLC SAX (see FIG. 3, drawing 3/4). The fraction with no affinity is constituted mainly of 1,6-anhydro derivatives, which, by their nature, cannot be converted into biotinylated derivatives.

By way of example, FIG. 3 describes the structure of certain main compounds of the oligosaccharide mixture. The referenced structures are represented below.

LC-MS analysis allows confirmation of the structure of these compounds via the mass spectra corresponding to the products in acid form: ΔIsIs_{Id1,6-anhydro} m/z=1056; ΔIsIs_{Id}Is_{Id1,6-anhydro} m/z=1633; ΔIsIs_{Id}Is_{Id}Is_{Id1,6-anhydro} m/z=2210; ΔIsIs_{Id} NH Biot m/z=1381; ΔIsIs_{Id}Is_{Id} NH Biot m/z=1958; ΔIsIs_{Id}Is_{Id}Is_{Id} NH Biot m/z=2535.

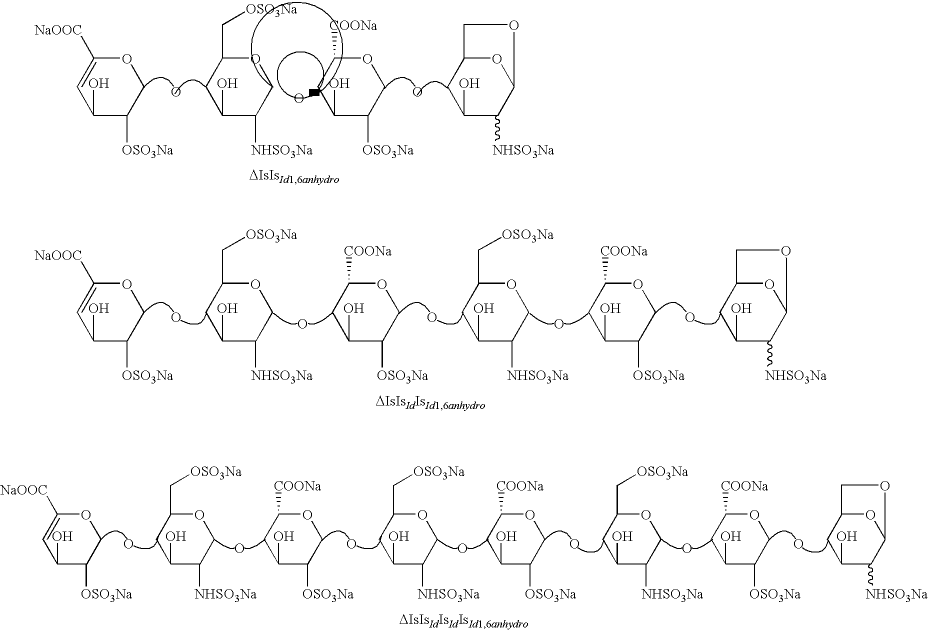

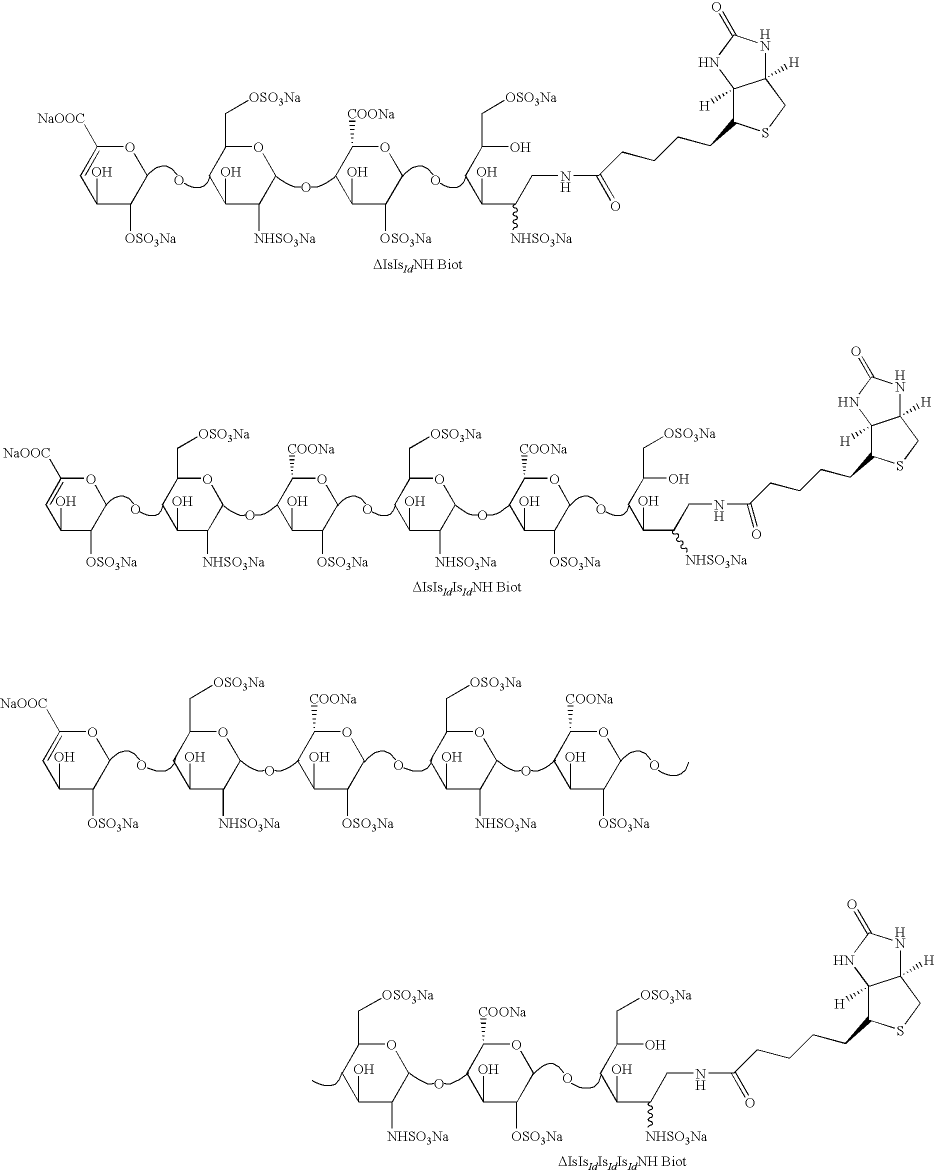

### Example 3

### NH-LC-LC Biotinoyl Enoxaparin

Enoxaparin, a low molecular weight heparin obtained according to the process described in U.S. Pat. RE38,743, may also be converted into a biotinylated derivative according to the reaction sequence described in scheme 4: enoxaparin is converted via a reductive amination reaction into compound 1 containing an amino function on its reducing end, and this derivative is then converted into the biotinylated compound 4 by reaction with the ester 3-sulfo-succinimidyl 6-biotinamidohexanoyl hexanoate, sodium salt.

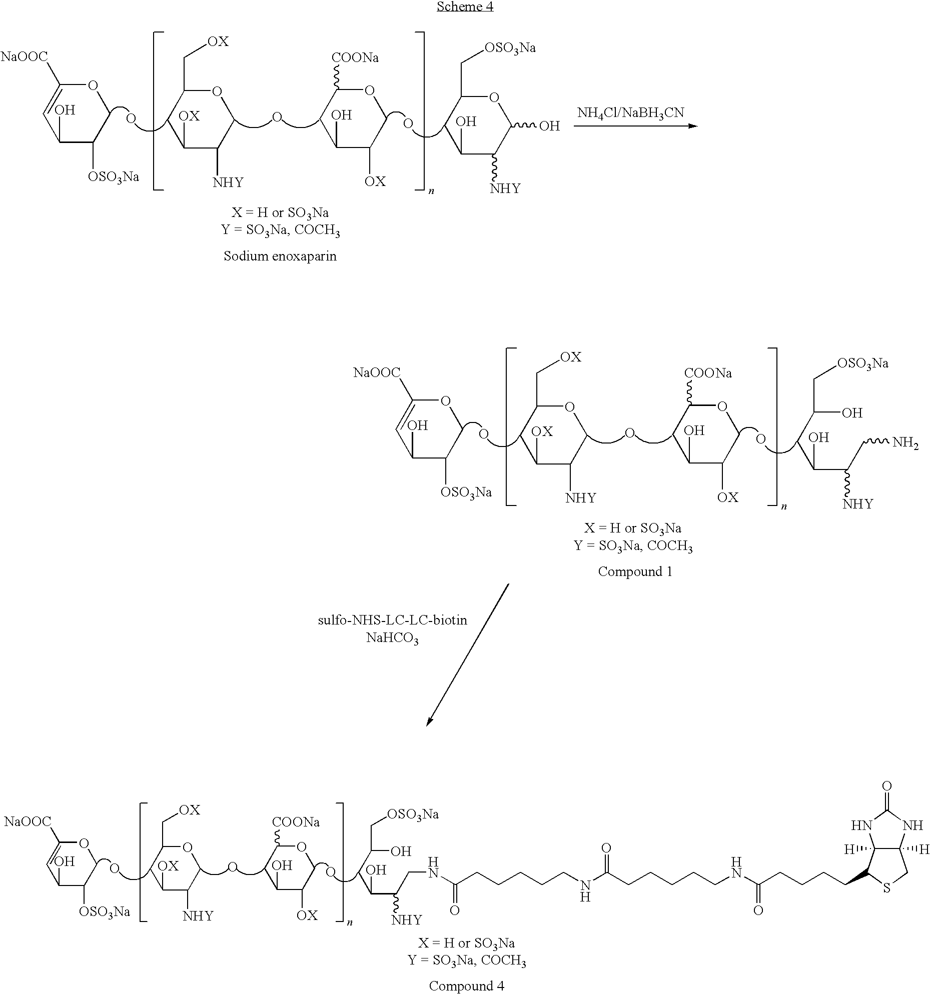

200 mg of 1-amino enoxaparin are dissolved in 5 ml of 0.5 M sodium hydrogen carbonate solution at a temperature in the region of 20° C. 164 mg of sulfo-NHS-LC-LC-biotin are added to the solution obtained. The solution is stirred at a temperature in the region 20° C. for 2 hours. The suspension is diluted with 10 ml of 0.5 M sodium hydrogen carbonate solution. 164 mg of sulfo-NHS-LC-LC-biotin are added and the mixture obtained is stirred for 5 hours. The reaction medium obtained is diluted with water (qs 150 ml), filtered through a 0.45 μm membrane and then injected onto a Q-Sepharose column. The product is eluted with water and then with a gradient of sodium perchlorate. The fraction collected is desalified on a column of Sephadex G10. The collected fraction is freeze-dried. 210 mg of a white lyophilizate are obtained. The observed yield is about 92%.

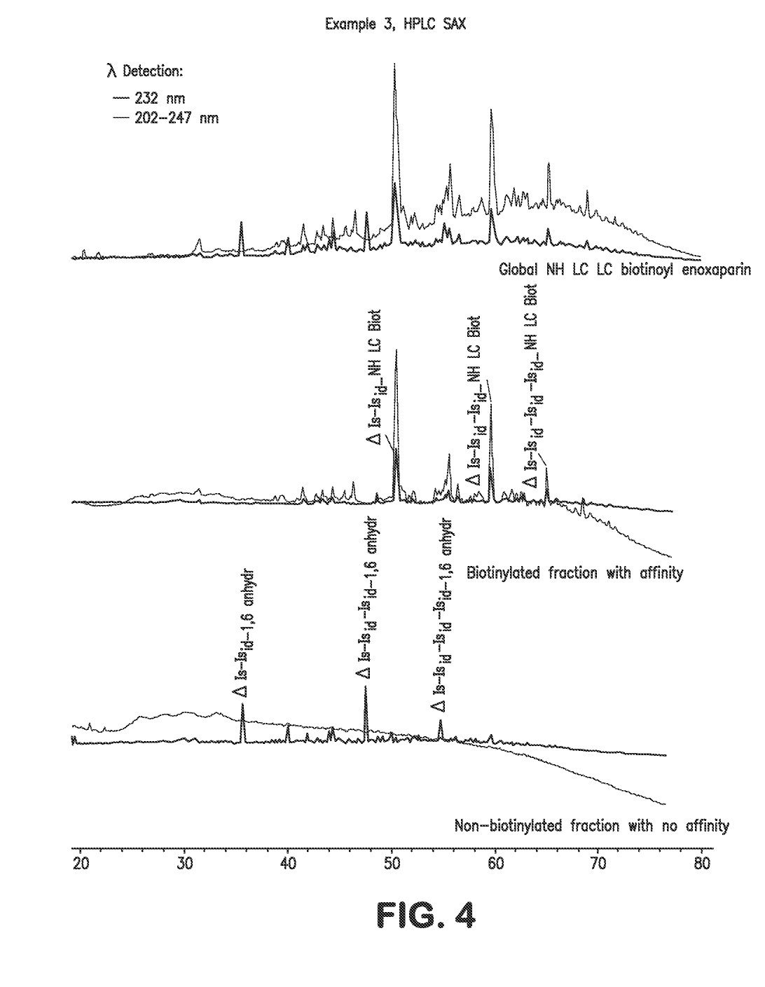

The product obtained is controlled by HPLC SAX (see FIG. 4, drawing 4/4, “Global” graph) and it is confirmed that the species containing an amino function on their reducing end are converted into the biotinylated derivative via reaction with 3-sulfosuccinimidyl 6-biotinamidohexanoyl hexanoate, sodium salt, in a degree of conversion of greater than 90%.

¹H NMR spectrum of the mixture of oligosaccharides in D₂O (25° C., δ in ppm): between 1.3 and 1.8 (16H, m), 2.05 (CH₃CO, s), 2.25 (6H, m), 2.80 (1H, dd, 12 and 7 Hz), 3.03 (1H, m), between 3.20 and 5.65 (polysaccharide protons), 5.98 (1H, d, 4 Hz).

The product obtained according to Example 3 is injected onto a supported avidin monomer column. Elution is performed according to the conditions described by the supplier Pierce. The biotinylated fractions (with affinity for avidin) and non-biotinylated fractions (with no affinity for avidin) obtained are then injected onto HPLC SAX (see FIG. 4). The fraction with no affinity is constituted mainly of 1,6-anhydro derivatives which, by their nature, cannot be converted into biotinylated derivatives.

The structure of the main compounds is confirmed by LC-MS coupling.

By way of example, FIG. 4 describes the structure of certain main compounds of the mixture of oligosaccharides. The referenced structures are represented below.

LC-MS analysis allows confirmation of the structure of the above compounds via the mass spectra corresponding to the products in acid form: ΔIsIs_{Id1,6-anhydro} m/z=1056; ΔIsIs_{Id}Is_{Id1,6-anhydro} m/z=1633; ΔIsIs_{Id}Is_{Id}Is_{Id1,6-anhydro} m/z=2210; ΔIsIs_{Id} NH LC LC Biot m/z=1607; ΔIsIs_{Id}Is_{Id} NH LC LC Biot m/z=2184; ΔIsIs_{Id}Is_{Id}Is_{Id} NH LC LC Biot m/z=2761.

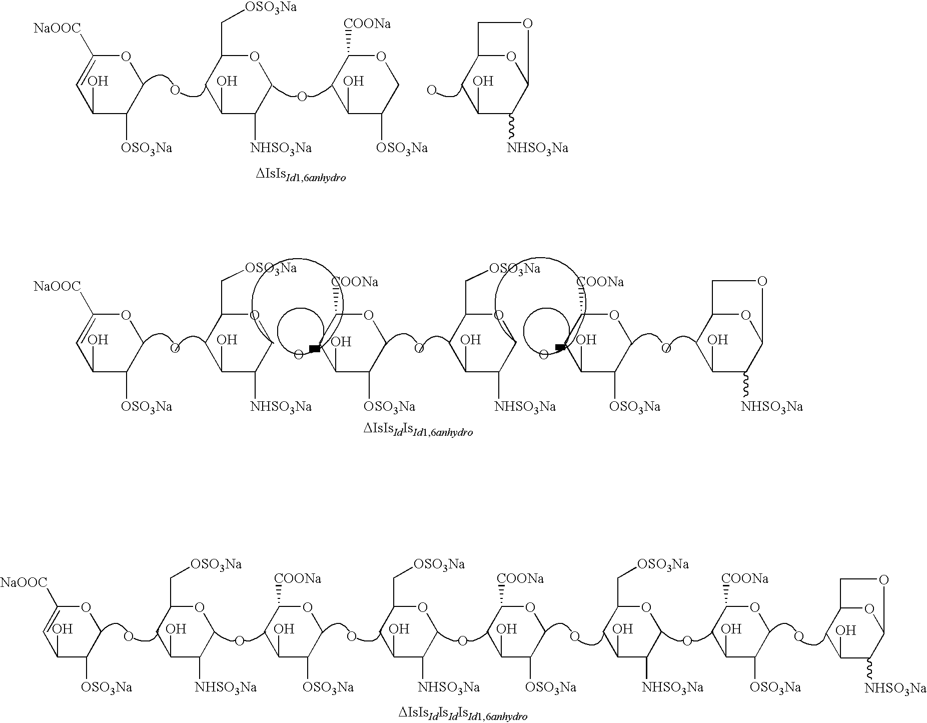

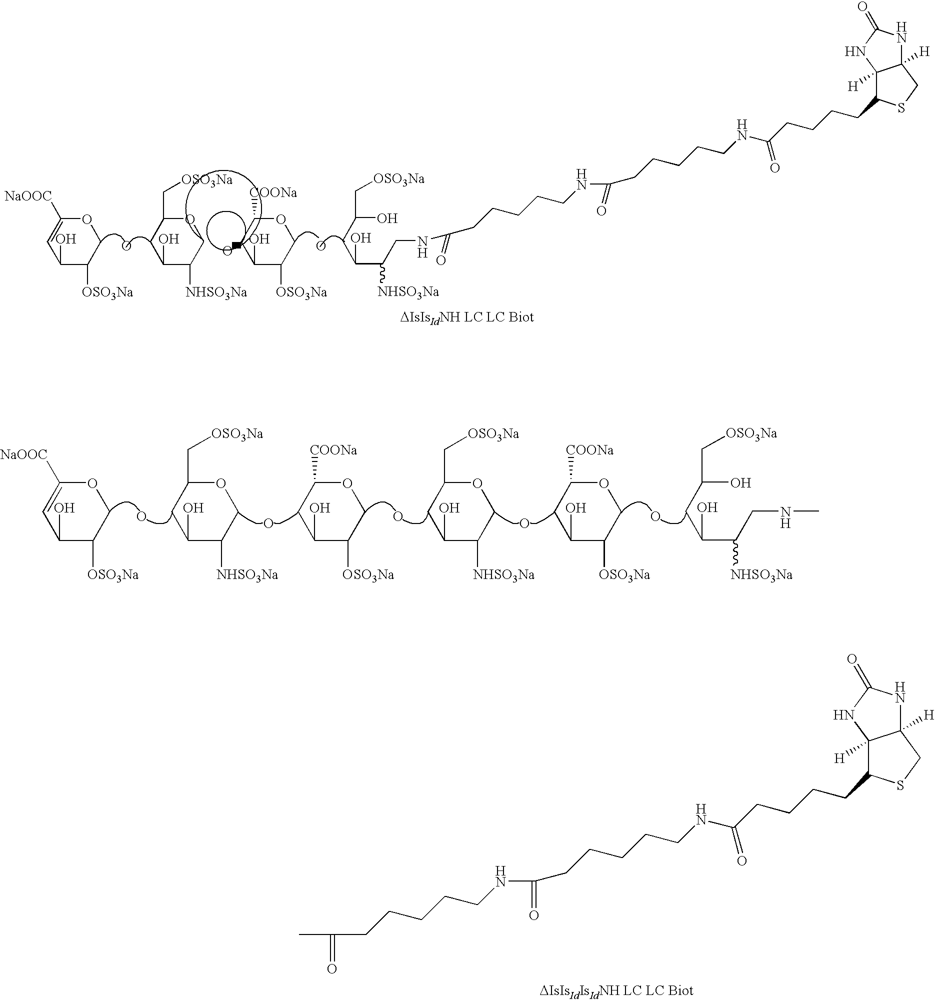

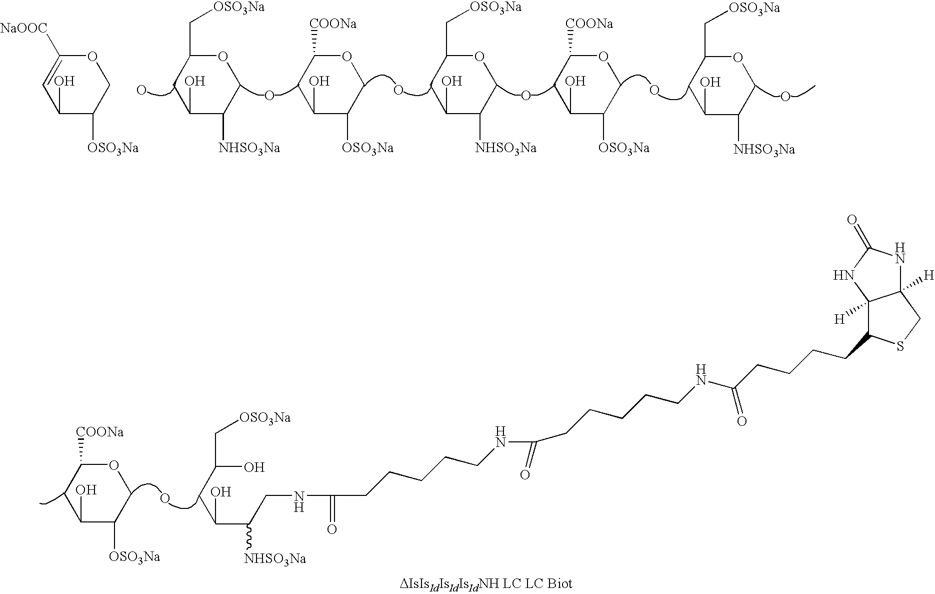

### Example 4

### NH-LC Biotinoyl Tinzaparin

Tinzaparin, a low molecular weight heparin of about 6000 daltons obtained by treatment with heparinase 1, may also be converted into a biotinylated derivative according to the reaction sequence described in Scheme 5: tinzaparin is converted via a reductive amination reaction into compound 5 containing an amino function on its reducing end, and this derivative is then converted into the biotinylated compound 6 via reaction with 3-sulfosuccinimidyl 6-biotinamido hexanoate, sodium salt.

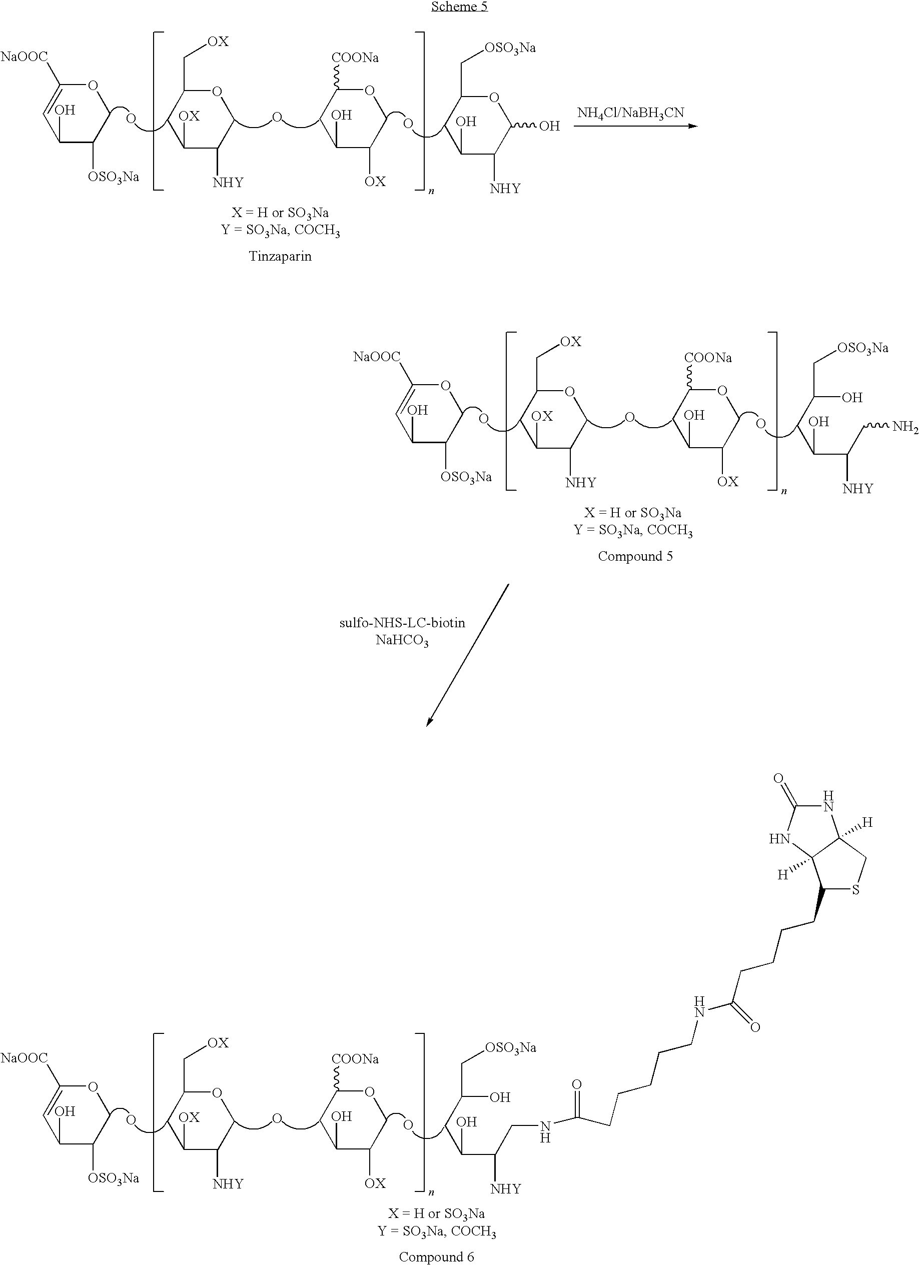

### 4.1: 1-Amino Tinzaparin

250 mg of tinzaparin are dissolved in 10 ml of aqueous 5 M ammonium chloride solution. 250 mg of sodium cyanoborohydride are added to the solution obtained. The mixture is maintained at 70° C. for 20 hours. The solution is cooled to a temperature in the region of 20° C. and diluted with water (qs 20 ml). The filtrate obtained is desalified on a column of Sephadex G10 and then freeze-dried. 215 mg of a white lyophilizate are obtained. The observed yield is 86%.

¹H NMR spectrum of the mixture of oligosaccharides in D₂O (25° C., δ in ppm): 2.05 (CH₃CO, s), 3.10 and 3.40 (1H each, m, CH₂NH₂), between 3.20 and 5.65 (polysaccharide protons), 5.98 (1H, d, 4 Hz).

The compound may be controlled by HPLC SAX, using the method outlined previously in Example 1.

The product is used without further purification in the biotinylation step.

### 4.2: NH LC Biotinoyl Tinzaparin

100 mg of 1-amino tinzaparin are dissolved in 2.5 ml of 0.5 M sodium hydrogen carbonate solution, at a temperature in the region of 20° C. 47 mg of sulfo-NHS-LC-biotin are added to the solution obtained. The solution is stirred at a temperature in the region of 20° C. for 1 hour 45 minutes. The suspension obtained is diluted with 5 ml of 0.5 M sodium hydrogen carbonate solution. 47 mg of sulfo-NHS-LC-biotin are added and the mixture obtained is stirred for 6 hours. A further 47 mg of sulfo-NHS-LC-biotin are added and the reaction mixture is stirred for 20 hours. The suspension is again diluted with 1 ml of 0.5 M sodium hydrogen carbonate solution and a further 47 mg of sulfo-NHS-LC-biotin are added. The reaction mixture is stirred for 20 hours. The suspension is again diluted with 6.5 ml of 0.5 M sodium hydrogen carbonate solution and a further 47 mg of sulfo-NHS-LC-biotin are added. The reaction mixture is stirred for 22 hours and then diluted with water (qs 100 ml), filtered through a 0.45 μm membrane and injected onto a column of Q-Sepharose. The product is eluted with water and then with a gradient of sodium perchlorate. The collected fraction is desalified on a column of Sephadex G10. The final fraction collected is freeze-dried. 110 mg of a white lyophilizate are obtained. The observed yield is quantitative.

¹H NMR spectrum of the mixture of oligosaccharides in D₂O (25° C., δ in ppm): between 1.3 and 1.8 (12H, m), 2.05 (CH₃CO, s), 2.25 (4H, m), 2.80 (1H, dd, 12 and 7 Hz), 3.03 (1H, m), between 3.20 and 5.65 (polysaccharide protons), 5.98 (1H, d, 4 Hz).

The compounds 1-amino tinzaparin and NH LC biotinoyl tinzaparin obtained may also be characterized via the HPLC SAX methods used previously in Example 1. This HPLC control shows that the species containing a functionalizable glucosamine are converted into a derivative containing an amino function on their reductive end, in a degree of conversion of greater than 90% to give 1-amino tinzaparin. It also shows that the species containing an amino function on their reducing end are converted into the biotinylated derivative via reaction with 3-sulfosuccinimidyl 6-biotinamido hexanoate, sodium salt, in a degree of conversion of greater than 90% to give NH LC biotinoyl tinzaparin.

In the same manner as in Example 1, the structures of the main compounds may be confirmed by LC-MS analysis.

The product obtained may also be injected onto a supported avidin monomer column. Elution is performed according to the conditions described by the supplier Pierce. The biotinylated fractions (with affinity for avidin) and non-biotinylated fractions (with no affinity for avidin) obtained may be controlled by HPLC SAX.

### Example 5

### NH LC Biotinoyl Bemiparin

Bemiparin, a low molecular weight heparin of about 3500 daltons, obtained via alkaline depolymerization, may also be converted into a biotinylated derivative according to the reaction sequence described in Scheme 6 below: bemiparin is converted via a reductive amination reaction into compound 7 containing an amino function on its reducing end, and this derivative is then converted into the biotinylated compound 8 via reaction with 3-sulfosuccinimidyl 6-biotinamido hexanoate, sodium salt.

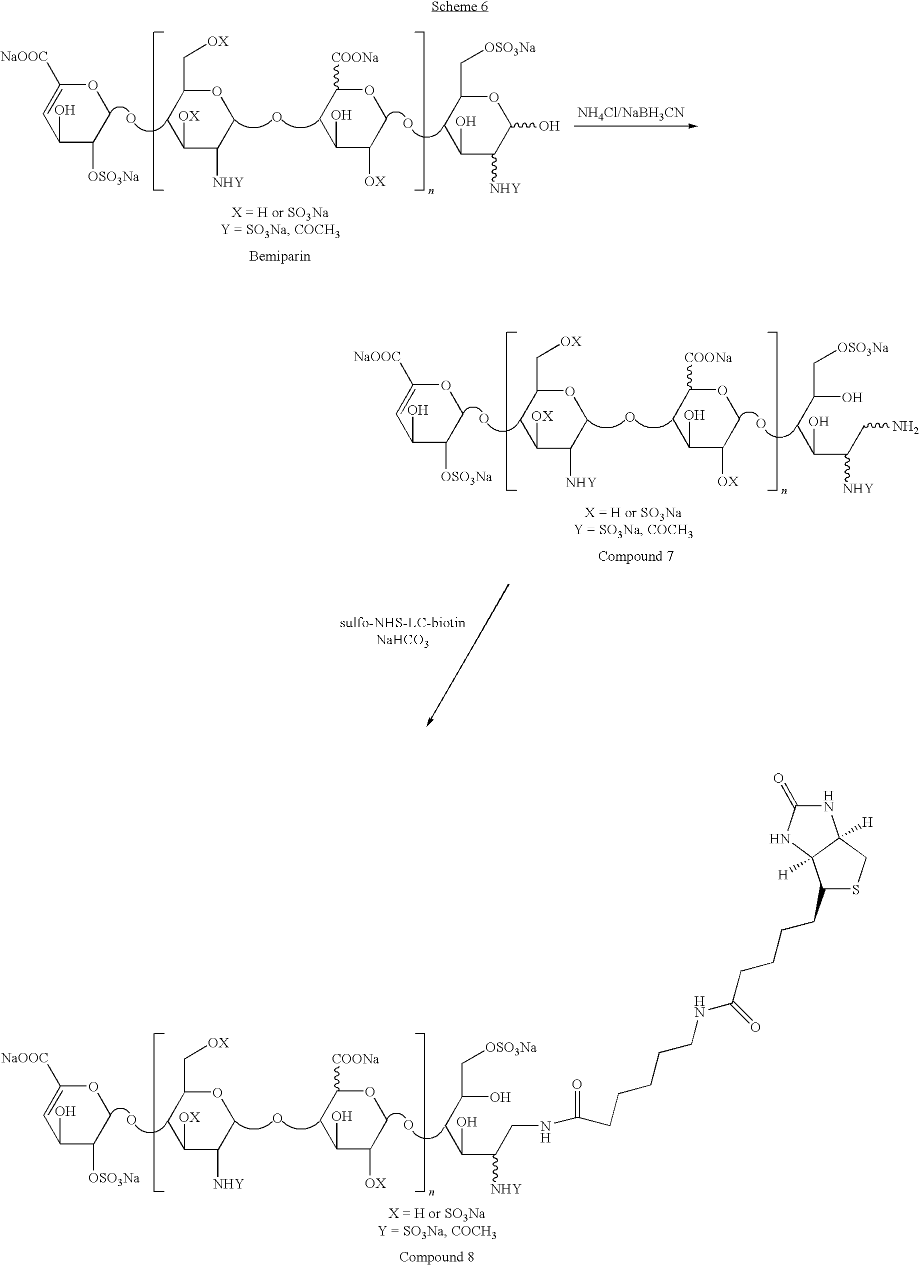

### 5.1: 1-Amino Bemiparin:

250 mg of bemiparin are dissolved in 10 ml of aqueous 5 M ammonium chloride solution. 250 mg of sodium cyanoborohydride are added to the solution obtained. The mixture is maintained at 70° C. for 20 hours. The solution is cooled to a temperature in the region of 20° C. and diluted with water (qs 20 ml). The solution obtained is desalified on a column of Sephadex G10 and then freeze-dried. 227 mg of a white lyophilizate are obtained. The observed yield is 91%.

¹H NMR spectrum of the mixture of oligosaccharides in D₂O (25° C., δ in ppm): 2.05 (CH₃CO, s), 3.10 and 3.40 (1H each, m, CH₂NH₂), between 3.20 and 5.80 (polysaccharide protons), 5.98 (1H, d, 4 Hz).

The compound may be controlled by HPLC SAX, using the method outlined previously in Example 1.

The product obtained is used without further purification in the biotinylation step.

### 5.2: NH LC Biotinoyl Bemiparin:

100 mg of 1-amino bemiparin are dissolved in 5 ml of 0.5 M sodium hydrogen carbonate solution, at a temperature in the region of 20° C. 80 mg of sulfo-NHS-LC-biotin are added to the solution obtained. The solution is stirred at a temperature in the region of 20° C. for 2 hours. The suspension obtained is diluted with 10 ml of 0.5 M sodium hydrogen carbonate solution. 80 mg of sulfo-NHS-LC-biotin are added and the mixture obtained is stirred for 2 hours. A further 40 mg of sulfo-NHS-LC-biotin are added and the reaction mixture is stirred for 20 hours. The reaction medium obtained is diluted with water (qs 50 ml) and then desalified on a column of Sephadex G10. The fraction obtained is injected onto a column of Q-Sepharose. The product is eluted with water and then with a gradient of sodium perrchlorate. The collected fraction is desalified on a column of Sephadex G10. The product obtained is again purified by passing it through a column of Q-Sepharose and desalified on Sephadex G10. The final fraction collected is freeze-dried. 101 mg of a white lyophilizate are obtained. The observed yield is 92%.

¹H NMR spectrum of the mixture of oligosaccharides in D₂O (25° C., δ in ppm): between 1.3 and 1.8 (12H, m), 2.05 (CH₃CO, s), 2.25 (4H, m), 2.80 (1H, dd, 12 and 7 Hz), 3.03 (1H, m), between 3.20 and 5.65 (polysaccharide protons), 5.98 (1H, d, 4 Hz).

The compounds 1-amino bemiparin and NH LC biotinoyl bemiparin obtained may also be characterized via the HPLC SAX methods used previously in Example 1. This HPLC control shows that the species containing a functionalizable glucosamine are converted into a derivative containing an amino function on their reducing end, in a degree of conversion of greater than 90% to give 1-amino bemiparin. It also shows that the species containing an amino function on their reducing end are converted into a biotinylated derivative via reaction with 3-sulfosuccinimidyl 6-biotinamido hexanoate, sodium salt, in a degree of conversion of greater than 90% to give NH LC biotinoyl bemiparin.

In the same manner as in Example 1, the structures of the main compounds may be confirmed by LC-MS analysis.

The product obtained may also be injected onto a supported avidin monomer column. Elution is performed according to the conditions described by the supplier Pierce. The biotinylated fractions (with affinity for avidin) and non-biotinylated fractions (with no affinity for avidin) obtained may be controlled by HPLC SAX.

The compounds according to the invention were subjected to biochemical and pharmacological studies.

### 1. Measurement of the Anti-Factor IIa Activity and of the Anti-Factor Xa Activity

The anti-factor IIa (anti-FIIa) activity and the anti-factor Xa (anti-FXa) activity in human plasma or a buffer system are analyzed via a chromogenic method: the anti-factor IIa activity is tested by means of the Actichrome heparin anti-factor IIa kit (American Diagnostica) containing the chromogenic substrate S-2238, α-thrombin and human ATIII (antithrombin III). The anti-FXa activity is determined with the automated coagulation instrument ACL 7000 (Instrumentation Laboratory) using the Heparin kit (Instrumentation Laboratory) containing ATIII, factor Xa and the chromogenic substrate S-2765. The two analyses are performed according to the manufacturer's instructions.

The following standards are used to establish a standard calibration curve for measuring the in vitro activity of the biotinylated low molecular weight heparin fractions in human plasma and the buffer system:
1st international standard for low molecular weight heparins (National Institute for Biological Standards and Control, London, UK, established in 1987, code No. 85/600),
2nd international standard for low molecular weight heparins (National Institute for Biological Standards and Control, London, UK, established in 1987, code No. 01/608, used since June 2006)
enoxaparin (Clexane®, sanofi-aventis, France) was used as internal reference.

For the determinations of anti-FIIa activity, 10 μl of sample or of international low molecular weight heparin standards are diluted to 1:16 with antithrombin in human plasma or the buffer system containing 0.05 M Tris HCl, 0.154 M NaCl, at pH 7.4. 10 μl of this solution are added to a 96-well microtitration plate. The measurement is repeated in triplicate (on 3 wells). The microtitration plate is maintained at 37° C. while agitating at 300 rpm. 40 μl of thrombin are added to each of the wells and incubated for exactly 2 minutes. 40 μl of Spectrozyme are added. After 90 seconds, the reaction is stopped by adding 40 μl of acetic acid. The absorption is measured at 405 nm using a SpectraMax 340 (Molecular Devices).

For the anti-FXa activity measurements, the sample or the international low molecular weight heparin standards are diluted in human plasma or the buffer system containing 0.05 M Tris HCl, 0.154 M NaCl, pH 7.4. The samples containing the heparinoids in the plasma or the buffer are again diluted to 1:20 with a working buffer containing ATIII, and placed in duplicate in the probe rotor. The factor Xa reagent and the chromogenic substrate are poured into the indicated reservoirs of the automated coagulation instrument ACL 7000.

The anti-FXa activity measurement is performed with the “heparin” protocol integrated into the ACL 7000 software. During the analysis, 50 μl of the sample (diluted with the working buffer) are mixed with 50 μl of the factor Xa reagent. After an incubation time of 60 seconds at 37° C., 50 μl of the chromogenic substrate of concentration 1.1 mM are added and the changes in absorption as a function of time are measured at a wavelength of 405 nm.

The results obtained are described especially in Table 1.

TABLE 1

| | | Anti-FXa activity | | Anti-FIIa activity | |
|---|---|---|---|---|---|
| | MM | (IU/mg) | | (IU/mg) | |
| | (Da) | measured | corrected | measured | corrected |
| Enoxaparin | 4100 | 121 | 121 | 28 | 28 |
| 1-Amino | 4100 | 116 | 116 | 26.5 | 26.5 |
| enoxaparin | | | | | |
| NH LC biotinoyl | 4441 | 101 | 109 | 21 | 22.7 |
| enoxaparin | | | | | |
| NH biotinoyl | 4328 | 98 | 103 | 29 | 31 |
| enoxaparin | | | | | |
| NH LC LC | 4653 | 82 | 93 | 24 | 27 |
| biotinoyl | | | | | |
| enoxaparin | | | | | |
| Tinzaparin | 6000 | 108 | 108 | 79 | 79 |
| 1-Amino | 6000 | 113 | 113 | 80 | 80 |
| tinzaparin | | | | | |
| NH LC biotinoyl | 6341 | 106 | 112 | 63 | 67 |
| tinzaparin | | | | | |
| Bemiparin | 3500 | 138 | 138 | 17 | 17 |
| 1-Amino | 3500 | 101 | 101 | 16 | 16 |
| bemiparin | | | | | |
| NH LC biotinoyl | 3841 | 93 | 102 | 13 | 14 |
| bemiparin | | | | | |

In this table, MM denotes the average molar mass (in daltons) and the “corrected” activity makes it possible to correct, in the measurement, the bulk dilution effect. The corrected activity is calculated as follows:

Corrected activity=(measured activity×MM prepared compound)/MM starting material,

with:
MM prepared compound: theoretical average molar mass of the prepared compound,
MM starting material: average molar mass of the starting low molecular weight heparin.

These results show that the biotinylated low molecular weight heparins according to the invention conserve anti-factor Xa and anti-factor IIa activities comparable to those of the native low molecular weight heparins. The conservation of these biological properties thus makes them therapeutically usable.

2. Measurement of the Anti-FXa Activity after Neutralization with Avidin
Neutralization of the Effect of Biotinylated Products with Avidin in Solution

The product-dependent anti-FXa or anti-FIIa antithrombin activity is measured in the presence of an increasing concentration of avidin in order to measure the effect of the binding of avidin to the biotin of the product on this activity.

The test products are dissolved at 1 mg/ml in water containing 0.9% NaCl. The products are then diluted so as to obtain a concentration of product capable of inhibiting 50% of the activity of factor Xa (Factor Xa, Chromogenix Milan, Italy) or of factor IIa (Factor IIa, laboratoire du sang [Blood Laboratory], Strasbourg) in the presence of antithrombin (human antithrombin, Milan, Italy). This inhibition is then measured in the presence of a decreasing concentration of avidin (Sigma avidin from egg white, Ref. A-9275, to be diluted in NaCl): 300, 30, 3, 0.3, 0.03, 0.003, 0 μg/ml. The assay of the residual activity of factor Xa (or factor IIa) is performed by adding a specific chromogenic substrate; S2222 (Chromogenix, Milan, Italy) for factor Xa and substrate S2238 (Chromogenix, Milan, Italy) for factor IIa. The optical density is read at 405 nm.

### Demonstration of the Binding of the Biotinylated Products to Avidin Bound to Beads in Buffer

In order to evaluate the avidin-binding capacity of the products, the products are placed in contact with avidin bound to beads. After centrifugation of the mixture, the anti-FXa or anti-FIIa activity is determined in the supernatant. This activity makes it possible to determine the concentration remaining in the medium and thus to determine the proportion of product trapped in the pellet after centrifugation of the mixture.

The test products are dissolved at 1 mg/ml in 0.9% NaCl solution. The products are diluted so as to be able to inhibit 80% of the anti-FXa or anti-FIIa activity present in the test. The bead solution is brought to 1 mg/ml by diluting it with the washing buffer 20 mM tris maleate, 150 mM NaCl, pH 7.35. The solution is agitated and 100 μl of the solution containing the beads (1 mg/ml) are placed in an Eppendorf tube. 500 μl of buffer are added. The tubes are centrifuged at 12 000 rpm for 5 minutes. After removal of the supernatant, the pellet is taken up in 500 μm of buffer. After agitation, a second centrifugation is performed and the supernatant is again discarded. The product solutions are then placed in contact with different solutions containing the beads so as to have the product/bead ratio expressed in μg of produit/μg of avidin (Sigma avidin Ref. A-9275, solution at about 3 mg/ml depending on the batch) of 1, 0.1, 0.01 and 0.001. The mixtures are then agitated and left to stand for 1 hour before centrifugation at 12 000 rpm for 5 minutes. The supernatant is then taken up to assay the anti-FXa activity in order to determine the concentration of product remaining in the supernatant. The anti-FXa or anti-FIIa activity is assayed by following a method modified from that described by Teien A. N and Lie M., Thrombosis Research, 1977, 10, 399-410. The results obtained are described especially in Table 2.

TABLE 2

| | Amount of avidin | |
|---|---|---|
| | (μg) | Residual anti-FXa activity |
| NH LC biotinoyl | 0.034 | 19% |
| enoxaparin | | |
| NH biotinoyl enoxaparin | 0.0245 | 19% |
| NH LC LC biotinoyl | 0.0276 | 13% |
| enoxaparin | | |

It is thus seen that the low molecular weight heparins have indeed been functionalized with biotin, to a degree of biotinylation of greater than 80%, and are indeed capable of being neutralized with avidin.

The biotinylated low molecular weight heparins according to the present invention may be used for the preparation of medicaments. They may especially be used as antithrombotic medicaments. Thus, according to another of its aspects, a subject of the invention is medicaments comprising a biotinylated low molecular weight heparin as defined above. These medicaments find their use in therapeutics, in particular in the treatment and prevention of venous thrombosis, arterial thrombotic accidents, especially in the case of myocardial infarction or unstable angina, peripheral arterial thrombosis, such as arteriopathy of the lower limbs, cerebral arterial thrombosis and strokes. They are also useful in the prevention and treatment of the proliferation of smooth muscle cells, angiogenesis, and as neuroprotective agents for atherosclerosis and arteriosclerosis.

According to another of its aspects, the present invention also relates to a method for treating the abovementioned pathologies, which comprises the administration to a patient of an effective dose of a compound according to the invention, or of a pharmaceutically acceptable salt thereof. The use of the biotinylated low molecular weight heparins as defined above for treating and preventing the above-mentioned pathologies thus forms part of the invention, as does the use of the said biotinylated low molecular weight heparins for the manufacture of a medicament for treating or preventing these pathologies.

According to another of its aspects, a subject of the present invention is a pharmaceutical composition comprising, as active principle, a biotinylated low molecular weight heparin according to the invention or a pharmaceutically acceptable salt thereof, and also at least one pharmaceutically acceptable inert excipient. The said excipients are chosen according to the desired pharmaceutical form and mode of administration, for example the oral, sublingual, subcutaneous, intramuscular, intravenous, transdermal, transmucosal, local or rectal route.

In each dosage unit, the active principle is present in the amounts suited to the envisaged daily doses in order to obtain the desired prophylactic or therapeutic effect. Each dosage unit may contain from 20 to 150 mg and advantageously from 40 to 100 mg of active principle. These doses of anticoagulant compounds may be neutralized with doses of avidin or of streptavidin ranging from 0.2 g to 2 g as an intravenous injection, bolus or infusion.

There may be special cases where higher or lower dosages are appropriate; such dosages are not outside the context of the invention. According to the usual practice, the dosage that is suitable for each patient is determined by the doctor according to the mode of administration and the weight and response of the said patient.

The compounds according to the invention may also be used in combination with one or more other active principles that are useful for the desired therapy, such as antithrombotic agents, anticoagulants or anti-platelet aggregating agents.

A subject of the present invention is also a process using avidin or streptavidin, characterized in that it makes it possible to neutralize the biotinylated low molecular weight heparins according to the invention. Thus, the avidin or streptavidin may be used for the preparation of medicaments for neutralizing the biotinylated low molecular weight heparins according to the present invention.

We claim:

1. A biotinylated low molecular weight heparin, comprising constituent polysaccharides having an average molecular weight of between 3000 and 7000 Da, wherein the constituent polysaccharides are covalently bonded to biotin or a biotin derivative at their reducing ends, or the pharmaceutically acceptable salts thereof.

2. The biotinylated low molecular weight heparin according to claim 1, wherein the covalently bonded constituent polysaccharides correspond to the formula (I): 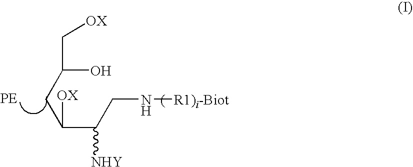
in which:
i is equal to 0 or 1,
R1 represents a sequence of formula (a) or (b): 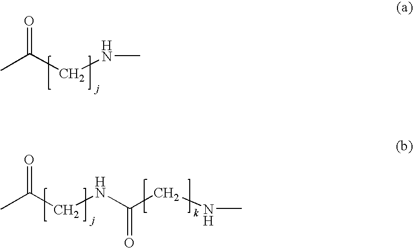
in which j and k, which may be identical or different, are integers that may take any value from 1 to 10,
Biot represents a biotin group or a biotin derivative,
PE represents a polysaccharide chain having the general structure of the constituent polysaccharides of heparin,
X represents H or SO₃Na,
Y represents SO₃Na or COCH₃, and
the wavy line denotes a bond located either below or above the plane of the pyranose ring to which it is attached.

3. The biotinylated low molecular weight heparin according to claim 2, wherein i is equal to 0.

4. The biotinylated low molecular weight heparin according to claim 2, wherein i is equal to 1 and R1 represents a sequence of formula (a) in which j is equal to 5.

5. The biotinylated low molecular weight heparin according to claim 2, wherein i is equal to 1 and R1 represents a sequence of formula (b) in which j and k are identical and are equal to 5.

6. The biotinylated low molecular weight heparin according to claim 2, wherein Biot represents the biotin group of formula (c):

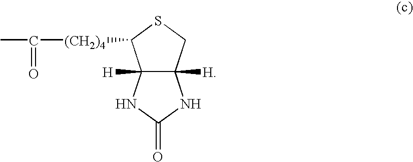

7. The biotinylated low molecular weight heparin according to claim 1, wherein at least 60% of the constituent polysaccharides have at their reducing ends a covalent bond to biotin or a biotin derivative.

8. The biotinylated low molecular weight heparin according to claim 7, wherein at least 80% of the constituent polysaccharides have at their reducing ends a covalent bond to biotin or a biotin derivative.

9. The biotinylated low molecular weight heparin according to claim 8, wherein at least 90% of the constituent polysaccharides have at their reducing ends a covalent bond to biotin or a biotin derivative.

10. The biotinylated low molecular weight heparin according to claim 1, wherein the low molecular weight heparin is chosen from enoxaparin, ardeparin, bemiparin, parnaparin and tinzaparin.

11. The biotinylated low molecular weight heparin according to claim 1, wherein the low molecular weight heparin is such that:
from 9% to 20% of its constituent polysaccharides have an average molecular weight of less than 2000 Da,
from 5% to 20% of its constituent polysaccharides have an average molecular weight of greater than 8000 Da,
from 60% to 86% of its constituent polysaccharides have an average molecular weight of between 2000 and 8000 Da,
the ratio between the mass-average molecular mass and the number-average molecular mass is between 1.3 and 1.6, and
the low molecular weight heparin has better bioavailability and antithrombotic activity than that of heparin and has an average molecular weight of approximately between 3500 and 5500 Da.

12. A process for preparing the biotinylated low molecular weight heparin as defined in claim 2, the process comprising the following steps:
a) performing a reductive amination on a low molecular weight heparin, in the presence of an amine salt and a reducing agent, at a temperature of between 20 and 80° C.; and
b) then performing an acylation with an activated group —(R1)ᵢ-Biot, in which R1, i and Biot are as defined in claim 2, in the presence of a base, in aqueous medium or in organic medium.

13. A process for preparing the biotinylated low molecular weight heparin as defined in claim 2, the process comprising the following steps:
a) performing a reductive amination on a low molecular weight heparin, in the presence of an ammonium halide salt and a borohydride salt, at a temperature of between 50 and 80° C., and
b) then performing an acylation with a group —(R1)ᵢ-Biot in activated ester form, in the presence of a base in aqueous medium.

14. A pharmaceutical composition comprising, as active principle, a biotinylated low molecular weight heparin according to claim 1 and at least one pharmaceutically acceptable excipient.

15. A method for the treatment or prevention of thrombosis in a patient, the method comprising administering to the patient an antithrombotic effective amount of a biotinylated low molecular weight heparin according to claim 1.

16. The method according to claim 15, wherein the biotinylated low molecular weight heparin is administered for treating and preventing venous thrombosis, arterial thrombotic accidents, peripheral arterial thrombosis, cerebral arterial thrombosis or strokes, the proliferation of smooth muscle cells, or angiogenesis, or as a neuroprotective agent for atherosclerosis and arteriosclerosis.

17. The method according to claim 16 wherein the arterial thrombotic accidents involve myocardial infarction or unstable angina, and the peripheral arterial thrombosis involves arteriopathy of the lower limbs.

18. A method for neutralizing the biotinylated low molecular weight heparin according to claim 1, the method comprising using a neutralizing amount of avidin or streptavidin

19. The method according to claim 15 further comprising administering avidin or streptavidin to neutralize the antithrombotic effect of the biotinylated low molecular weight heparin.
